(19) United States
(12) Patent Application Publication
(10) Pub. No.: US 2025/0288637 A1
(43) Pub. Date: Sep. 18, 2025

(54) BLACK SEED OIL FORMULATIONS

(71) Applicant: Novatek Therapeutics, LLC, Pearland, TX (US)

(72) Inventors: Mohamed KASEB, Pearland, TX (US); Abdulhafez SELIM, Pearland, TX (US); Ahmed Omar KASEB, Pearland, TX (US); Steve PONDELL, Pearland, TX (US)

(73) Assignee: Novatek Therapeutics, LLC, Pearland, TX (US)

(21) Appl. No.: 18/861,428

(22) PCT Filed: May 1, 2023
(86) PCT No.: PCT/US2023/066465

§ 371 Date: Oct. 29, 2024

Related U.S. Application Data
(60) Provisional application No. 63/336,749, filed on Apr. 29, 2022.

(51) Int. Cl. A61K 36/71; A61K 9/48

(52) U.S. Cl. CPC A61K 36/71; A61K 9/4816; A61K 9/4866; A61K 9/4891

(57) ABSTRACT

A black seed oil formulation that includes an enteric capsule and black seed oil contained therein for administration to a subject.

### FIELD OF THE INVENTION

The presently disclosed subject matter relates to black seed oil formulations.

### BACKGROUND

Black seed oil has been observed to have many beneficial heath effects, such as reduction of hypertension, inflammation, allergies, asthma, and anti-viral and anti-cancer properties. Unfortunately, some patients who administer black seed oil have reported negative gastric side effects. Accordingly, there remains a need for new way to administer black seed oil, while reducing negative gastric side effects.

### BRIEF SUMMARY

One aspect of the subject disclosure provides a black seed oil formulation that includes an enteric capsule and black seed oil contained therein. In one exemplary embodiment, the enteric capsule comprises a coating that includes an enteric component applied thereon. In an alternative or further embodiment, the enteric capsule includes an enteric component incorporated directly into the capsule itself (e.g., incorporated into the substrate layer of the capsule).

In an exemplary embodiment, the enteric capsule includes an acid-insoluble polymer. In an exemplary embodiment, the enteric capsule includes a film-forming polymer. In an exemplary embodiment, the enteric capsule includes hydroxypropyl methylcellulose phthalate (HPMCP) and/or the enteric capsule includes a hydroxypropyl methylcellulose (HPMC) substrate layer.

In an exemplary embodiment, the black seed oil formulation includes a banding solution applied thereon. The banding solution, in exemplary embodiments, includes a second enteric component. In one particular embodiment, the enteric component (e.g., HPMCP) and the second enteric component (e.g., HPMCP) are the same.

In an exemplary embodiment, the black seed oil contains at least 0.25 wt %, or at least 0.5 wt %, or at least 0.75 wt %, or at least 1 wt %, or at least 1.25 wt %, or at least 1.5 wt %, at least 1.6 wt %, or at least 1.75 wt %, at least 2 wt %, at least 2.1 wt %, or at least or 2.5 wt %, based on the total weight of black seed oil in the formulation.

In one exemplary embodiment, the black seed oil formulation remains intact in gastric acid (e.g., endogenous or simulated gastric acid) having a pH 3 or lower. Accordingly, when administered to a subject, the black seed oil formulation does not dissolve or disintegrate in the stomach of the subject, thereby avoiding or alleviating gastric discomfort that can occur upon administering other black seed oil formulations.

In one exemplary embodiment, the black seed oil formulation dissolves or disintegrates at a pH greater than about 5.5. Accordingly, when administered to a subject, the black seed oil formulation is released lower in the digestive tract, e.g., in the duodenum, in the jejunum and/or in the colon. For example, the black seed oil formulation, in an exemplary embodiment, dissolves or disintegrates at a pH of about 6 to about 7, while remaining intact at a pH under about 5.5 to target the jejunum or dissolves or disintegrates at a pH of above 7, while remaining intact at a pH under about 6.0 for ileum and colon targeted delivery.

In one exemplary embodiment, a black seed oil formulation is provided that includes an enteric capsule comprising a hydroxypropyl methylcellulose (HPMC) substrate layer with an enteric coating comprising hydroxypropyl methylcellulose phthalate (HPMCP); and black seed oil contained in the enteric capsule, the black seed oil comprising at least 1.5 wt % thymoquinone, based on the total weight of black seed oil in the formulation. In one embodiment, the black seed oil further includes a banding solution applied thereon, the banding solution comprising hydroxypropyl methylcellulose phthalate (HPMCP). In one exemplary embodiment, the black seed oil is in an amount of about 500 mg, though other amounts can be provided.

### DETAILED DESCRIPTION

The invention can be more fully appreciated by reference to the following description, including the examples. Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In addition, the materials, methods, and examples are illustrative only and not intended to be limiting.

### Terms and Definitions

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value. Alternatively, with respect to biological systems or processes, the term “about” can mean within an order of magnitude, within 5-fold, or within 2-fold on either side of a value. Numerical quantities given herein are approximate unless stated otherwise, meaning that the term “about” or “approximately” can be inferred when not expressly stated.

To provide a more concise description, some of the quantitative expressions given herein are not qualified with the term “about.” It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity for which that could be obtained under the particular stoichiometric conditions. Concentrations that are given as percentages refer to mass ratios, unless indicated differently.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof, unless limitation to the singular is explicitly stated.

The terms “comprising” and “including” are used herein in their open, non-limiting sense. Other terms and phrases used in this document, and variations thereof, unless otherwise expressly stated, should be construed as open ended, as opposed to limiting. Thus, the term “example” is used to provide exemplary instances of the item in discussion, not an exhaustive or limiting list thereof. Similarly, adjectives such as “conventional,” “traditional,” “normal,” “criterion,” “known,” and terms of similar meaning should not be construed as limiting the item described to a given time period or to an item available as of a given time, but they should be read to encompass conventional, traditional, normal, or criterion technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. As will become apparent to one of ordinary skill in the art after reading this document, the illustrated embodiments and their various alternatives may be implemented without confinement to the illustrated examples.

The term “carrier” refers to an adjuvant, vehicle, or excipients, with which the compound is administered. In certain embodiments, the carrier is a solid carrier. Suitable pharmaceutical carriers include those described in Remington: The Science and Practice of Pharmacy, 21^{st} Ed., Lippincott Williams & Wilkins (2005).

The term “formulation,” as used herein, is the form in which the dose is to be administered to the subject or patient. The black seed oil extract can be administered as part of a formulation that includes non-active agents, such as an enteric (e.g., enteric coated) capsule.

The term “pharmaceutically acceptable,” as used in connection with formulations of the subject disclosure, refers to molecular entities and other ingredients of such formulations that are physiologically tolerable and do not typically produce untoward reactions when administered to an animal (e.g., human) according to their intended mode of administration (i.e., oral).

A “pharmaceutically acceptable excipient” refers to a substance that is non-toxic, biologically tolerable, and otherwise biologically suitable for administration to a subject, such as an inert substance, added to a pharmacological formulations or otherwise used as a vehicle, carrier, or diluents to facilitate administration of an agent and that is compatible therewith. Suitable pharmaceutical carriers include those described in Remington: The Science and Practice of Pharmacy, 21^{st} Ed., Lippincott Williams & Wilkins (2005).

As used herein, the term “inert” refer to any inactive ingredient of a described formulation. The definition of “inactive ingredient” as used herein follows that of the U.S. Food and Drug Administration, as defined in 21 C.F.R. 201.3(b)(8), which is any component of a drug product other than the active ingredient.

As used herein, “suitable for oral administration” refers to a sterile, pharmaceutical product, such as a product produced under good manufacturing practices (GMP), as understood in the art, suitable for administration to a subject (e.g., a human subject).

As used herein, the term “disorder” is used interchangeably with “disease” or “condition”. For example, a pulmonary disorder also means a pulmonary disease or a pulmonary condition.

The terms “treat,” “treating,” and “treatment” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. These terms also include ameliorating a symptom of a disease (e.g., reducing the pain, discomfort, or deficit), wherein such amelioration may be directly affecting the disease (e.g., affecting the disease's cause, transmission, or expression) or not directly affecting the disease.

As used in the present disclosure, the term “effective amount” is interchangeable with “therapeutically effective amount” and means an amount or dose of thymoquinone, and/or other active components in the black seed oil, effective in treating the particular disease, condition, or disorder disclosed herein, and thus “treating” includes producing a desired preventative, inhibitory, relieving, or ameliorative effect. In methods of treatment according to the invention, “an effective amount” of any one of the presently described formulations is administered to a subject (e.g., a mammal). The “effective amount” will vary, depending on the compound, the disease (and its severity), the treatment desired, age and weight of the subject, etc. and can be determined by persons of ordinary skill in the art based on the circumstance.

The terms “individual,” “subject,” and “patient” are used interchangeably herein and can be a vertebrate, in particular, a mammal, more particularly, a primate (including non-human primates and humans) and include a laboratory animal in the context of a clinical trial or screening or activity experiment. Thus, as can be readily understood by one of ordinary skill in the art, the formulations of the present invention are particularly suited to administration to any vertebrate, particularly a mammal, and more particularly, a human.

Reference will now be made to the embodiments of the present invention, examples of which are illustrated by and described in conjunction with the accompanying examples. While certain embodiments are described herein, it is understood that the described embodiments are not intended to limit the scope of the invention. On the contrary, the present disclosure is intended to cover alternatives, modifications, and equivalents that can be included within the invention as defined by the appended claims.

In exemplary embodiments, the black seed oil formulations of the subject disclosure are formulated in a capsule that, as finally formulated for oral administration, is stable (i.e., does not dissolve) at pH commonly found in the stomach (e.g., pH of about 3 or lower), but readily breaks down (i.e., dissolves or disintegrates) at a higher pH commonly found in the small intestine and further downstream in the digestive tract (e.g., pH of about 5.5 to about 9). It has been previously suggested that having substantially all the drug release at pH of >5.5 (e.g., 90% solubility of the formulation at pH 6.8), but not at lower pH, may increase the bioavailability of the black seed oil and targeting delivery of the drug to the intestine region, and thus may find applications in particular indications, such as irritable bowel syndrome. See. e.g., Azad et al., Encapsulation of Black Seed Oil in Alginate Beads as a pH-Sensitive Carrier for Intestine-Targeted Drug Delivery: In Vitro, In Vivo and Ex Vivo Study, Pharmaceutics 2020, 12(3), 219, hereby incorporated by reference.

More particularly, the formulations of the instant disclosure can allow for the formulation to remain intact and not dissolve or disintegrate at low pH as it enters the stomach (e.g., pH of about 3 or lower), but that dissolves at pH's to target the duodenum, jejunum or ileum and colon. For example, the formulations, in certain embodiments, can dissolve at pH >5.5 (duodenum targeting), or pH 6-7 (jejunum targeting), or pH above 7 (ileum and colon targeting), while remaining intact and/or not dissolving or disintegrating at lower pHs conditions that are found further upstream in the digestive tract. This can be achieved, for example, based on the capsule, enteric coating and/or banding solution employed. As used herein, and for purposes of brevity, the term “enteric” refers to any one of such formulations.

In certain embodiments, whether a formulation is deemed to be intact, or dissolved or disintegrated, is determined by USP <711>: Dissolution, in either or both of Apparatus 1 (Basket Stirring Element) and/or Apparatus 2 (Paddle Stirring Element).

In embodiments, the instantly disclosed black seed oil formulations are formulated to provide the instantly disclosed enteric properties via the use of a capsule that is itself enteric, or via the use of a capsule that is initially not enteric, but has been modified with an enteric coating prior to loading the black seed oil, so as to provide an enteric formulation as administered by the subject. In such embodiments, the enteric coating that is applied the capsule can also serve as the banding solution.

Alternatively, a banding solution can be separately applied as a separate step.

Examples of enteric coatings that can find use according to the subject disclosure include enteric components known in the art, including acid-insoluble polymers and film-forming polymers. In exemplary embodiments, the acid-insoluble polymer can also be selected from the group consisting of acrylic and methacrylic acid copolymers, cellulose acetate esters such as phthalate, butyrate, hydroxypropyl methylcellulose phthalate, and salts thereof. In exemplary embodiments, the film-forming polymer is selected from the group consisting of cellulose acetate phthalate, cellulose acetate tremellitate, HPMCP, hydroxy propyl methyl cellulose acetate succinate (HPMCAS), polyvinyl acetate phthalate (PVAP), and methacrylic acid copolymers.

For example, HPMCP is a phthalic acid ester of hydroxypropyl methylcellulose and has been admitted into the U.S. National Formulary (US/NF). Capsules in which enteric coatings such as HPMCP have been applied are commercially available from, for example, CapsCanada® (Dania Beach, FL), SE Tylose GmbH & Co. KG (Wiesbaden, Germany).

The threshold pH at which, for example, an enteric capsule that includes HPMCP, will dissolve can be controlled, for example, by varying the phthalyl content. In certain exemplary embodiments, the formulation dissolves at, for example, pH >5.5 (duodenum targeting), or pH 6-7 (jejunum targeting), or pH above 7 (ileum and colon targeting).

Another enteric coating that can find use according to the subject disclosure include coatings that include a polymer having methyl acrylate as a monomer (e.g., a copolymer of methyl acrylate) commercialized with different acidic or alkaline groups to allow for the formulation to remain intact and not dissolve or disintegrate at low pH, but that dissolves at, for example, pH >5.5 (duodenum targeting), or pH 6-7 (jejunum targeting), or pH above 7 (ileum and colon targeting). Methyl acrylate enteric coatings, as described above are commercially available (e.g., Eudagrit® Polymers for Delayed Release such as Eudragit® L 30 D-55, Eudragit® FS 30 D, Eudragit® L and Eudragit® S polymers, commercially available from Evonik Industries AG (Essen, Germany).

In certain exemplary embodiments, a hard capsule is provided. Alternatively, in certain exemplary embodiments, a soft capsule is provided. In either case, in exemplary embodiments, the surface of a pre-manufactured capsule is coated (e.g., spraying or film-coating already-manufactured capsules) with one or more layers of a substance or composition that is known to impart enteric properties, such as, but not limited to, a composition that includes HPMCP or a copolymer of methyl acrylate. Alternatively, in other exemplary embodiments, enteric components (e.g., HPMCP, copolymers of methyl acrylate, and other acid-insoluble polymers) are incorporated directly into the hard or soft capsule upon their initial manufacture (i.e., introducing the enteric polymer upon initially preparing the hard or soft capsules). Thus, in this technique, the impartation of the enteric properties occurs during the manufacturing process as opposed to treating capsules which have already been pre-formed.

In certain exemplary embodiments, film-forming polymers, known to those of ordinary skill in the art, can also be incorporated to provide an enteric coating in the instantly disclosed black seed oil formulations.

A banding solution can be applied to the formulations of the present disclosure. Conventionally, capsules contain two halves. One half of the capsule if loaded with formulation containing black seed oil and then the other half of the capsule is engaged with the loaded half to encapsulate the formulation. A banding solution can be applied to the joined capsule to facilitate continued and long-term engagement of the capsule. In certain embodiments, the banding solution contains an enteric component, which can be the same enteric component incorporated into the enteric coating and/or into the capsule itself.

In certain embodiments, the amount of thymoquinone present in the administered formulation of black seed oil is at least 0.25 wt %, or at least 0.5 wt %, or at least 0.75 wt %, or at least 1 wt %, or at least 1.25 wt %, or at least 1.5 wt %, at least 1.6 wt %, or at least 1.75 wt %, at least 2 wt %, at least 2.1 wt %, or at least or 2.5 wt %, based on the total weight of black seed oil in the formulation. Dosing amounts of the black seed oil can be adjusted based on the concentration of thymoquinone in the administered black seed oil formulation.

The presently disclosed black seed oil formulations can be administered to treat any indication or condition in a subject, or administered to healthy subjects seeking to maintain their good health. In certain exemplary embodiments, the presently disclosed formulations are administered to a subject (e.g., orally to a human subject) to treat an inflammatory disease or condition. For example, the presently disclosed formulations can be administered to treat an inflammatory disease or condition selected from allergy, asthma, COPD, autoimmune diseases, celiac disease, colitis, irritable bowel syndrome, intestinal hyperplasia, metabolic syndrome, obesity, diabetes, rheumatoid arthritis, liver disease, hepatic steatosis, fatty liver disease, non-alcoholic fatty liver disease, and non-alcoholic steatohepatitis, glomerulonephritis, hepatitis, inflammatory bowel disease, reperfusion injury and transplant rejection.

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Oral Black Seed Oil Formulation

Black seed oil (BSO) is loaded into size 00 HPMC hard shell, enteric capsules (500 mg BSO/capsule) commercially available from CapsCanada® (Dania Beach, FL) under the tradename AR-CAPS®. The HPMC capsules are provided with a hydroxypropyl methylcellulose phthalate (HPMCP) coating to provide the enteric capsule. The capsules are provided in two pieces, the black seed oil is loaded into the capsules, and the two pieces are joined together. In a separate operation, a liquid formulation of HPMCP is applied as a banding solution to the area where the top and bottom of the capsule meet in order to seal the capsule. The filled and sealed capsules are then inspected for leakers and dried.

For the sake of brevity, all publications, including patent applications, patents, and other citations mentioned herein, are incorporated by reference in their entirety to the same extent as if each individual publication, patent or patent application was specifically and individually incorporated by reference. Citation of any such publication, however, shall not be construed as an admission that it is prior art to the present invention.

While this invention has been particularly shown and described with references to preferred embodiments thereof, it will be understood by those skilled in the art that various changes in form and details can be made therein without departing from the scope of the invention encompassed by the claims. Further, all embodiments included herein are given solely for the purpose of illustration and are not to be construed as limitations of the present invention, as many variations thereof are possible without departing from the spirit and scope of the invention.

1. A black seed oil formulation comprising an enteric capsule and black seed oil contained therein.

2. The black seed oil formulation of claim 1, wherein the enteric capsule comprises a coating that includes an enteric component applied thereon.

3. The black seed oil formulation of claim 1, wherein the enteric capsule includes an enteric component incorporated directly into the capsule.

4. The black seed oil formulation of claim 1, wherein the enteric capsule includes an acid-insoluble polymer.

5. The black seed oil formulation of claim 1, wherein the enteric capsule includes a film-forming polymer.

6. The black seed oil formulation of claim 1, wherein the enteric capsule includes hydroxypropyl methylcellulose phthalate (HPMCP).

7. The black seed oil formulation of claim 1, wherein the enteric capsule includes a hydroxypropyl methylcellulose (HPMC) substrate layer.

8. The black seed oil formulation of claim 2 or 3, further comprising a banding solution applied thereon.

9. The black seed oil formulation of claim 8, wherein the banding solution comprises a second enteric component.

10. The black seed oil formulation of claim 9, wherein the enteric component and the second enteric component are the same.

11. The black seed oil formulation of claim 10, wherein the enteric component is hydroxypropyl methylcellulose phthalate (HPMCP).

12. The black seed oil formulation of claim 1, wherein the black seed oil contains at least 1.5 wt % thymoquinone, based on the total weight of blackseed oil in the formulation.

13. The black seed oil formulation of claim 1, wherein the black seed oil contains at least 2 wt % thymoquinone, based on the total weight of blackseed oil in the formulation.

14. The black seed oil formulation of claim 1, wherein the formulation remains intact in gastric acid having a pH 3 or lower.

15. The black seed oil formulation of claim 12, wherein the formulation dissolves or disintegrates at a pH greater than about 5.5.

16. The black seed oil formulation of claim 12, wherein the formulation dissolves or disintegrates at a pH of about 6 to about 7, while remaining intact at a pH under about 5.5.

17. The black seed oil formulation of claim 12, wherein the formulation dissolves or disintegrates at a pH of above 7, while remaining intact at a pH under about 6.0.

18. A black seed oil formulation comprising:
an enteric capsule comprising a hydroxypropyl methylcellulose (HPMC) substrate layer with an enteric coating comprising hydroxypropyl methylcellulose phthalate (HPMCP); and
black seed oil contained in the enteric capsule, the black seed oil comprising at least 1.5 wt % thymoquinone, based on the total weight of black seed oil in the formulation.

19. The blackseed oil formulation of claim 18, further comprising a banding solution applied thereon, the banding solution comprising hydroxypropyl methylcellulose phthalate (HPMCP).

20. The black seed oil formulation of claim 18 or 19, wherein the black seed oil is in an amount of about 500 mg.
